(19) United States
(12) Patent Application Publication
(10) Pub. No.: US 2026/0301921 A1
(43) Pub. Date: Oct. 1, 2026

(54) SYSTEMS AND METHODS FOR IMPROVING CLINICAL PHOTOGRAPHY CAPTURE AND WORKFLOW

(71) Applicant: VANDERBILT UNIVERSITY, Nashville, TN (US)

(72) Inventors: Michael GOLINKO, Nashville, TN (US); Mark ARRIETA, Nashville, TN (US); Michael CAUDILL, Nashville, TN (US); Brandon OGLESBY, Nashville, TN (US); Peter SHAVE, Nashville, TN (US); Michael SIMARD, Nashville, TN (US); Alex WINSTON, Nashville, TN (US)

(21) Appl. No.: 19/475,088

(22) PCT Filed: Apr. 22, 2024
(86) PCT No.: PCT/US24/25737

§ 371 Date: Oct. 14, 2025

Related U.S. Application Data
(60) Provisional application No. 63/497,611, filed on Apr. 21, 2023.

(51) Int. Cl. G16H 30/20; G16H 10/60

(52) U.S. Cl. CPC G16H 30/20; G16H 10/60

(57) ABSTRACT

A medical image capture (MIC) computing device captures standardized photographs for clinical and non-clinical applications. Access to medical records is provided to a software application of a user's device. The user's device captures multiple views of the user based on one or more standards. During the image capture process, the user is prompted to position and align themselves within key elements of the view desired. The standardized medical photographs may be used to assist with medical intervention, consultations, operations, and evaluations.

### CROSS-REFERENCE TO RELATED APPLICATIONS

This application claims priority to U.S. 63/497,611, filed Apr. 23, 2023, which is hereby incorporated by reference as submitted in its entirety.

### BACKGROUND

### Field

The present disclosure relates to medical photography and, more particularly, to systems and methods for capturing standardized photographs for clinical and non-clinical use.

### BACKGROUND

Facial images play a crucial role in various aspects of clinical medicine including documentation of and healing from injury, accurate assessment and planning prior to operative and non-operative interventions, in addition to intraoperative guidance and documentation. Moreover, each patient's response to any intervention at any given time is unique, and medical images help tailor the approach to their specific needs.

Preoperative Planning and Assessment: Facial Reconstruction: Before performing medical plastic surgery, surgeons rely on detailed images to assess the patient's features, proportions, and any existing deformities or asymmetries. These images help them plan the surgical approach, determine incision locations, and anticipate potential challenges and moreover are available as objective evidence of change in the subject over time.

Computer Imaging: Surgeons often use computer software to manipulate images, simulating the expected outcomes of procedures such as rhinoplasty (nose reshaping), facelifts, or eyelid surgery. Patients can visualize the potential changes and discuss their preferences with the surgeon.

Multimodal Imaging: Combining various imaging techniques (such as 3D scans, CT scans, and photography) provides a comprehensive picture of the patient's anatomy, aiding in precise surgical planning.

Intraoperative Guidance and Documentation: Surgical Navigation: During surgery, real-time imaging (such as intraoperative CT scans or 3D models) assists surgeons in navigating delicate facial structures. It ensures accurate placement of implants, grafts, or sutures.

Medicolegal Documentation: High-quality medical photographs are essential for legal purposes, documenting the patient's condition before and after surgery (or even non-surgical intervention, such as injection of fillers or botox). These images serve as evidence of the clinical outcome and can be crucial in case of disputes or claims.

Monitoring Progress: Post-intervention images track the healing process, scar formation, and overall aesthetic results. Surgeons can adjust their management based on visual feedback, ensuring optimal outcomes.

Scientific Dissemination: Accurate and reproducible standardized images of the face are requisite for dissemination to the scientific community in peer-reviewed journals and or meeting presentations. Without some standard to present clinical images, audiences' have little confidence to draw meaningful conclusions from images presented.

Currently, there are challenges related to standardization of photos in both clinical and non-clinical contexts including standardization challenges, intuitive and reproducible capture, and secure integration with medical records. The lack of standardization when capturing medical photos poses significant challenges. For example, clinicians encounter difficulties in achieving consistent and reproducible results due to variations in techniques and equipment. Additionally, clear protocols are essential to ensure standardized practices across different scenarios. The prior art lacks an intuitive, elegant, and reproducible way to capture a standardized medical image. Additionally, the prior art lacks a seamless way to securely store captured images in a medical record, such as an electronic health record associated with a patient. Prior solutions require the use of cumbersome equipment, special backgrounds, space, and trained staff to capture the standardized photos. FIG. 1 illustrates a prior art solution requiring consistent angles, orientation, special lighting, and a special background. Currently, there is a lack of seamless integration between standardized medical photos and medical records and finding efficient ways to incorporate these photos into patient records remains a challenge.

### BRIEF SUMMARY

The present embodiments may relate to, inter alia, systems and methods for providing systems and methods for clinical photography capture. The systems and methods include, for example, an intuitive and elegant method for capturing standardized medical images. The method may, for example, provide clinicians with a straightforward process that ensures reproducibility across patients, settings, and users. Additionally, clinicians may adapt techniques for specific disciplines, allowing for personalization while maintaining standardization. The present embodiments provide seamless integration between standardized medical photos and medical records. Additionally, the present embodiments overcome resource constraints (e.g., equipment, background, space, and staff).

In one aspect, a medical image capture (MIC) computing device may be provided. The MIC computing device may capture standardized face photographs for a plurality of medical providers and a plurality of patients. The MIC computing device may include one or more processors that are configured to: (i) coordinate access to one or more medical records between one or more medical record servers and a medical provider of the plurality of medical providers, wherein each of the one or more medical records is associated with each of the plurality of patients, (ii) receive, from a computing device associated with the medical provider, a first request for image data of a patient of the plurality of patients, (iii) identify, based on the first request, one or more views required in accordance with a procedure identified by the first request, (iv) transmit, to a computing device of the patient, a second request for one or more photographs in accordance with the one or more views, and (v) receive, from the computing device of the patient, captured image data of the patient.

In a second aspect, a method for capturing standardized face photographs for a plurality of medical providers and a plurality of patients may be provided. The method may include: (i) coordinating access to one or more medical records between one or more medical record servers and a medical provider of the plurality of medical providers, wherein each of the one or more medical records is associated with each of the plurality of patients, (ii) receiving, from a computing device associated with the medical provider, a first request for image data of a patient of the plurality of patients, (iii) identifying, based on the first request, one or more views required in accordance with a procedure identified by the first request, (iv) transmitting, to a computing device of the patient, a second request for one or more photographs in accordance with the one or more views, and (v) receiving, from the computing device of the patient, captured image data of the patient.

In a third aspect, a non-transitory computer-readable medium comprising instructions may be provided. The instructions, when executed by one or more processors, may implement a method for capturing standardized face photographs for a plurality of medical providers and a plurality of patients may be provided. The method implemented by the instructions may include: (i) coordinating access to one or more medical records between one or more medical record servers and a medical provider of the plurality of medical providers, wherein each of the one or more medical records is associated with each of the plurality of patients, (ii) receiving, from a computing device associated with the medical provider, a first request for image data of a patient of the plurality of patients, (iii) identifying, based on the first request, one or more views required in accordance with a procedure identified by the first request, (iv) transmitting, to a computing device of the patient, a second request for one or more photographs in accordance with the one or more views, and (v) receiving, from the computing device of the patient, captured image data of the patient.

Advantages will become more apparent to those skilled in the art from the following description of the preferred embodiments which have been shown and described by way of illustration. As will be realized, the present embodiments may be capable of other and different embodiments, and their details are capable of modification in various respects. Accordingly, the drawings and description are to be regarded as illustrative in nature and not as restrictive.

### BRIEF DESCRIPTION OF THE DRAWINGS

The Figures described below depict various aspects of the systems and methods disclosed therein. It should be understood that each Figure depicts an embodiment of a particular aspect of the disclosed systems and methods, and that each of the Figures is intended to accord with a possible embodiment thereof. Further, wherever possible, the following description refers to the reference numerals included in the following Figures, in which features depicted in multiple Figures are designated with consistent reference numerals.

For a detailed description of various examples, reference will now be made to the accompanying drawings in which:

FIG. 1 shows a prior art diagram of a cumbersome environment for taking clinical photographs.

FIG. 2 shows an exemplary medical image capture computing system in accordance with one or more embodiments.

FIG. 3A depicts an exemplary method 300A for capturing medical image data of a patient in accordance with one or more embodiments.

FIG. 3B depicts an exemplary method 300B for image capture in accordance with one or more embodiments.

FIG. 4 shows exemplary medical image views of a patient in accordance with one or more embodiments.

FIGS. 5A-5I show exemplary screenshots provided by one or more interfaces provided by a software application to capture medical images in accordance with one or more embodiments.

FIG. 6 shows a simplified system diagram for an electronic device in accordance with one or more embodiments.

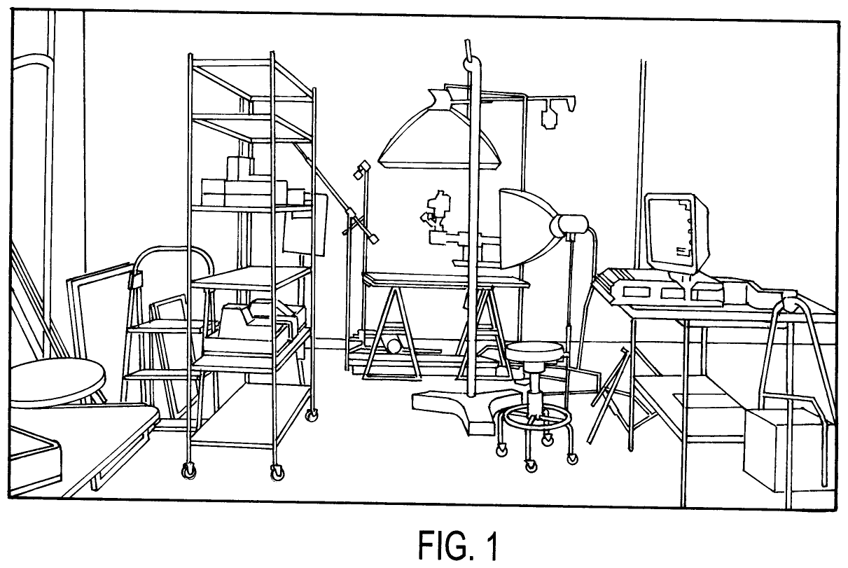

The Figures depict example embodiments for purposes of illustration only. One skilled in the art will readily recognize from the following discussion that alternative embodiments of the systems and methods illustrated herein may be employed without departing from the principles of the embodiments described herein.

### DETAILED DESCRIPTION

The subject matter will now be descried in detail for specific preferred embodiments. It is understood that the described embodiments are intended only as illustrative examples and are not to be limited thereto.

The present embodiments may relate to, inter alia, systems and methods for medical image capture. In some embodiments, an intuitive and elegant method for capturing standardized medical images is provided. The method may, for example, provide users, such as clinicians, with a straightforward process that ensures reproducibility across patients and settings. Additionally, clinicians may adapt techniques for specific disciplines, allowing for personalization while maintaining standardization. Further, the systems and methods described herein provide seamless integration with a patient's medical health record.

In some embodiments of the disclosed technology, systems and methods for medical image capture using a mobile device are provided. The mobile device may be used to capture standardized medical images of an individual. For example, a software application on the mobile device may provide a user interface that may guide the user through a medical image capture process. The medical image capture process may, via the user interface, provide the user with graphical indicators to assist the user with capturing photos that meet medical image standards.

In some embodiments of the disclosed technology, systems and methods for medical image capture may include the capturing of standardized medical images of an individual, such as a patient. The standardization of medical images may be based on one or more guidelines established by one or more governing bodies, such as the American Society of Plastic Surgeons (ASPS), or the like. Guidelines for medical image capture may include, for example, target area, reproduction ratio, patient distance, patient preparation, patient positioning, or the like. Guidelines for standardized medical photography may be provided for different areas of the body (e.g., close-up face, full face, ears, mouth).

In some embodiments of the disclosed technology, systems and methods for medical image capture using one or more dynamic digital templates is provided. For example, during an image capture process, one or more graphical indicators, or dynamic digital templates (e.g., arrows, image overlays, dashed lines) may be shown on the screen of a mobile device indicating how the patient should position their body part to be photographed, such as their face (e.g., looking up/down or left/right) as an overlay of the area within the camera's field of view. The dynamic digital templates may, in some embodiments, help align the part of the patient (e.g., the patient's nose) that is to be photographed. The dynamic digital templates assist with aligning the patient consistently throughout the image capture process. During image capture, the dynamic digital template may change colors indicating whether the patient is either in alignment or not in alignment. For example, before the patient is in alignment, the dynamic digital template may be shown in red. Once in alignment, the dynamic digital template may change from red to green.

In some embodiments of the disclosed technology, systems and methods for medical image capture may include seamless integration of the medical image capture process with a patient's electronic medical record. In some embodiments, the integration may be performed in accordance with one or more standards and/or privacy rules (e.g., HIPAA Privacy Rule) used to protect and secure individuals' medical records. In some embodiments, captured medical images may be securely stored, either locally or on a remote storage device, and shared with trusted users (e.g., medical professionals that have been granted access to the captured images). Additionally, or alternatively, captured medical images may be uploaded to a user's electronic medical record. Notifications may be triggered when images are uploaded. For example, a notification may be issued to a medical professional informing them of updated patient data. In another example, a notification may be issued to the patient indicating the upload status of captured medical image data (e.g., confirmation or indication of progress). Further, captured images may be uploaded directly from a software application on the patient's computing device (e.g., mobile phone) to the patient's electronic medical record.

In some embodiments, the disclosed technology may be used across multiple disciplines, including clinical and non-clinical settings.

Medical use cases for the disclosed technology may be used for assistance and improvement in medical decision making but also documentation in peer-review print and meeting settings. Example medical use cases include:
Any surgical or non-surgical intervention on the face to examine and document before and after and longitudinal photos over time.
Any facial injury that needs documentation of the injury (e.g., laceration, burn, bruise, fracture) that documents healing, resolution or complication over time.
Longitudinal documentation of any facial feature, such as a mole (e.g., watch for concerning features of cancer), hair loss skin tone or color.
Documentation of growth of a child's face or documentation of a pathologic process of the face, such as a tumor, cyst, fibrous dysplasia over time that may need intervention.
General use in telehealth medicine for all of the above purposes: patients can send in standardized-“selfies” or images for review by the clinical team.
Documentation of professional and amateur athlete-patients, of injury, recovery and training progress.

Alternatively, non-medical use cases for the disclosed technology may be applicable. Example non-medical use cases include:
Governmental and non-government photo employee ID badges and identification documents, making it more secure and standardized.
Use of photo documentation in the penal/judiciary system.
Higher education student ID's.
Health, beauty fitness applications to track changes in the body over time from interventions (e.g., diet plans, exercise regimens) or barbers and hair salons to document a customer's hair style over time.

The above use-cases are in no way meant to be limiting, but merely provided as an example for understanding the disclosure.

At least one of the technical problems addressed by this system may include: (i) lack of standardization of medical photos for clinical and non-clinical applications, (ii) lack of intuitive, elegant and reproducible ways to capture a standardized medical image, (iii) lack of seamless integration between standardized medical photos and medical records, and (iv) having to use cumbersome equipment, use special backgrounds, use up valuable space, and having to train staff to capture standardized photos.

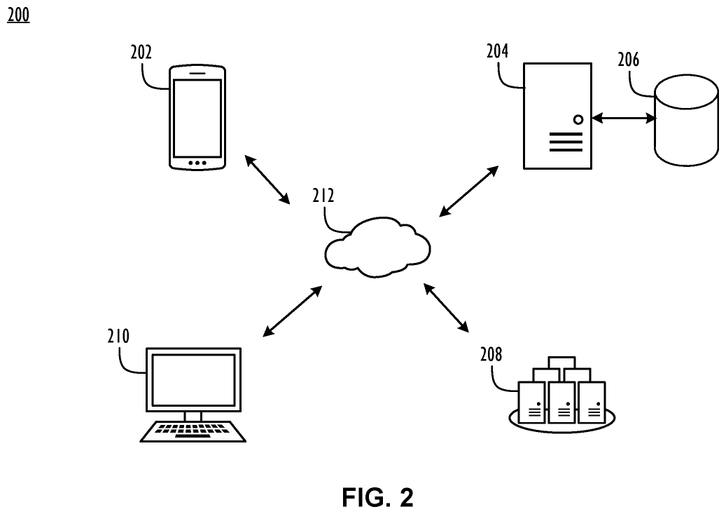

Exemplary Computing System for Capturing Standardized Photos FIG. 2 depicts an exemplary medical image capture (MIC) system 200. MIC system 200 may include a MIC computing device 204. MIC computing device 204 may comprise, for example, a database server that may be communicatively coupled to a database 206 that stores data. In one embodiment, database 206 may be a local storage device. In another embodiment, database 206 may be a remote storage device, such as cloud storage system, or the like. MIC computing device 204 may be in communication with a plurality of user devices accessed by patients and medical providers to interface with the MIC system 200, such as user devices 202 and 210.

Further, MIC computing device 204 may be in communication with a plurality of medical record servers 208. In some embodiments, medical record servers 208 may store digital patient information within the physical premises of a healthcare provider (e.g., hospitals, clinics, or private practices). In this example, healthcare organizations have full control over their data, including security measures and access permissions. Additionally, some institutions prefer on-premises solutions to comply with specific regulations or privacy requirements. Alternatively, medical record servers 208 may store, manage, and process digital patient information using remote storage solutions, such as cloud computing solutions. In some embodiments, cloud servers can easily scale up or down based on demand, accommodating growing patient data. Medical providers may pay for the resources they use, avoiding upfront hardware costs. Additionally, authorized users may access patient records securely from anywhere with an internet connection. For example, users, via mobile device 202 or computer device 210, may access medical record servers 208 via network 212. A cloud-based server may facilitate data sharing among healthcare providers, improving collaboration. In some embodiments, a combination of on-premises and cloud-based medical record storage may be implemented. For example, an on-premises storage device may periodically synchronize with a cloud-based server. Additionally, or alternatively, the cloud-based server may synchronize with the on-premises storage device in response to a triggering event (e.g., when a patient uploads new data or medical images to their electronic medical record).

In some exemplary embodiments, user devices 202 and 210 may be computers that include a web browser or a software application, to access remote computer devices, such as MIC computing device 204, using the Internet or other network 212. More specifically, user devices 202 and 210 may be communicatively coupled to MIC computing device 204 through many interfaces including, but not limited to, at least one of the Internet, a network, such as the Internet, a local area network (LAN), a wide area network (WAN), or an integrated services digital network (ISDN), a dial-up-connection, a digital subscriber line (DSL), a cellular phone connection, and a cable modem. User devices 202 and 210 may be any device capable of accessing the Internet including, but not limited to, a desktop computer, a laptop computer, a personal digital assistant (PDA), a cellular phone, a smartphone, a tablet, a phablet, wearable electronics, smart watch, or other web-based connectable equipment or mobile devices. User devices 202 and 210 may include GPS sensors, accelerometers, and gyroscope, as described herein. MIC computing device 204 may receive user data (e.g., medical data, medical image data) from user devices 202 and 210. Further, MIC computing device 204 may securely store the collected user data on database 206.

In the exemplary embodiment, medical record servers 208 may comprise computers that include a web browser or a software application, which enables medical record servers 208 to access remote computer devices, such as MIC computing device 204, using the Internet or other network. More specifically, provider devices may be communicatively coupled to MIC computing device 204 through many interfaces including, but not limited to, at least one of the Internet, a network, such as the Internet, a local area network (LAN), a wide area network (WAN), or an integrated services digital network (ISDN), a dial-up-connection, a digital subscriber line (DSL), a cellular phone connection, and a cable modem. Medical record servers 208 may include any device capable of accessing the Internet including, but not limited to, a desktop computer, a laptop computer, a personal digital assistant (PDA), a cellular phone, a smartphone, a tablet, a phablet, wearable electronics, smart watch, or other web-based connectable equipment or mobile devices.

In some embodiments, medical record servers 208 may serve as an intermediary between MIC computing device 204 and a third-party server (not shown). Medical record servers 208 may be used to provide compensation to users (e.g., associated with user devices 202 or 210). More specifically, medical record servers 208 may be communicatively coupled to the Internet through many interfaces including, but not limited to, at least one of a network, such as the Internet, a local area network (LAN), a wide area network (WAN), or an integrated services digital network (ISDN), a dial-up-connection, a digital subscriber line (DSL), a cellular phone connection, and a cable modem.

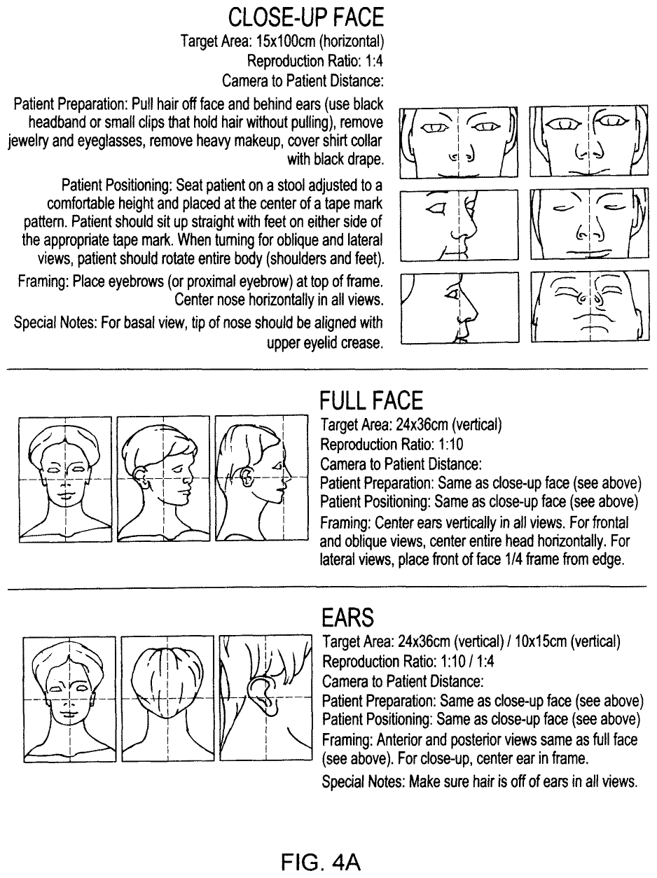

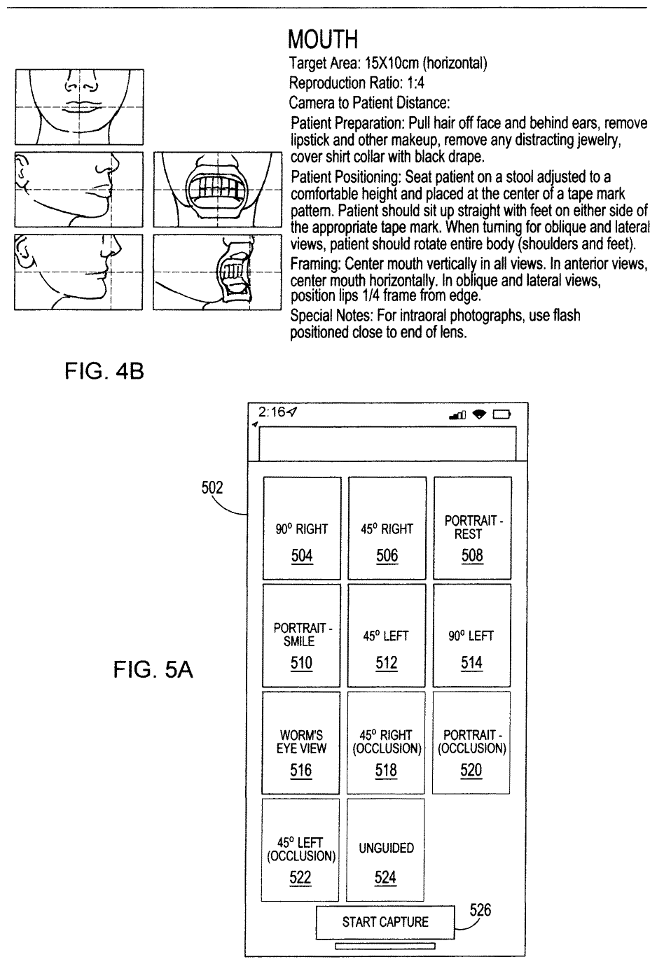

MIC computing device 204 may receive medical image data standards from medical record servers 208. The received data standards may originate from a governing body, such as ASPS or the like. The received data standards may detail requirements for medical image capture of a patient. Detail requirements may include, for example, required views of a patient (e.g., front, side, perspective, close-up). Additionally, or alternatively, detail requirements may include capture capability requirements of an image capture device for capturing medical image data of a patient. For example, the image capture device may have to provide a certain level of image quality (e.g., number of megapixels, exposure settings). In another example, detail requirements may include standards required for a specific procedure. For example, in one embodiment, medical providers associated with MIC computing device 204 may only be interested in images required for certain procedures (e.g., views of the nose for nose surgery or views of the eyes for eyelid surgery). Accordingly, the medical providers, through MIC computing device 204, may specify: 1) type of surgery, 2) image quality level requirements, and 3) minimum image views. In some embodiments, the medical provider may only be required to indicate the type of surgery being performed and MIC computing device 204 may match the type of surgery with one or more standards. Based on the standards, the image quality level requirements and minimum image views required may be determined. Example views of a patient are illustrated in FIG. 4. As shown, views may include view 402 (close-up face), view 404 (full face), view 406 (ears), and view 408 (mouth). Additional, or less, views may be required, depending on the type of procedure being performed as described herein. Each view may have one or more parameters or descriptors, such as target area, reproduction ratio, camera-to-patient distance, patient preparation, patient positioning, framing, special notes, or the like. Additionally, the one or more standards may define views for specific body parts, such as a patient's nose, neck, forearm, or the like.

### Exemplary Method for Capturing Medical Image Data

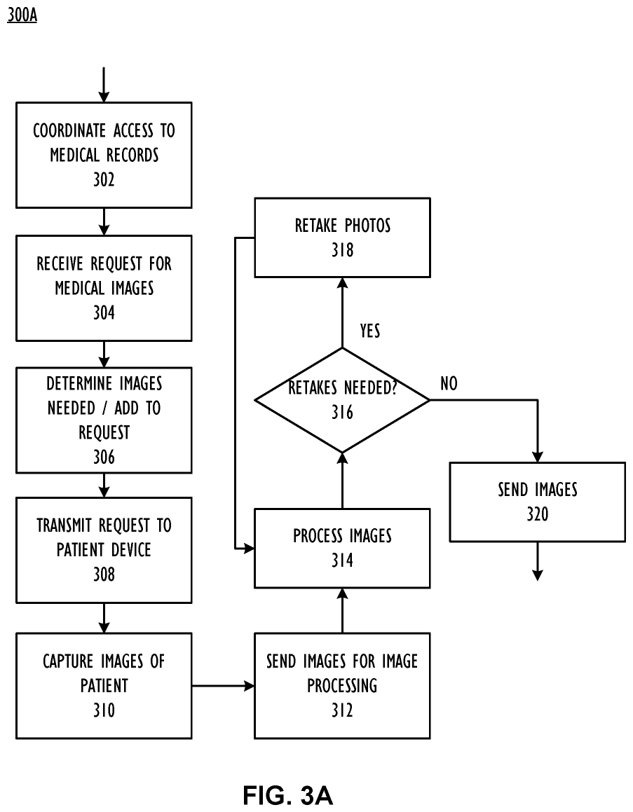

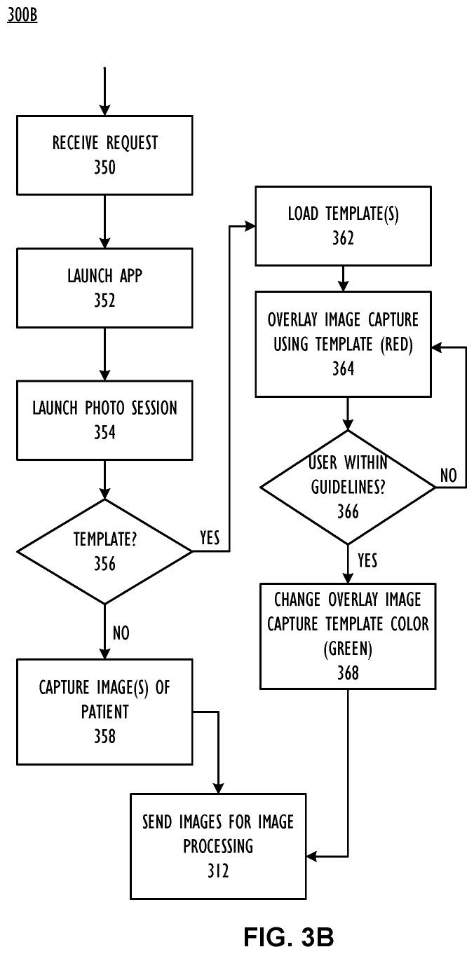

FIG. 3A depicts an exemplary method 300A for capturing medical image data of a patient, processing and verifying the captured images, and distributing the captured images among medical providers, electronic medical record providers, or the like. Method 300A may be performed by MCI computing device 204 (shown in FIG. 2). Further, steps of method 300A may be performed in conjunction with one or more user devices 202 and 210 and medical provider devices 208, as shown in the exemplary configuration illustrated in FIG. 2.

Method 300A may include coordinating 302, by the MIC computing device 204 with medical record servers 208, to share electronic medical records associated with patients. For example, medical record servers 208 may store an electronic medical record associated with a patient that is associated with user device 202. Authorization may be provided by the patient for a medical provider, such as a medical provider associated with user device 210, to access the patient's electronic medical record. Access may include, for example, full access (e.g., read and write access) or limited access (e.g., read-only access).

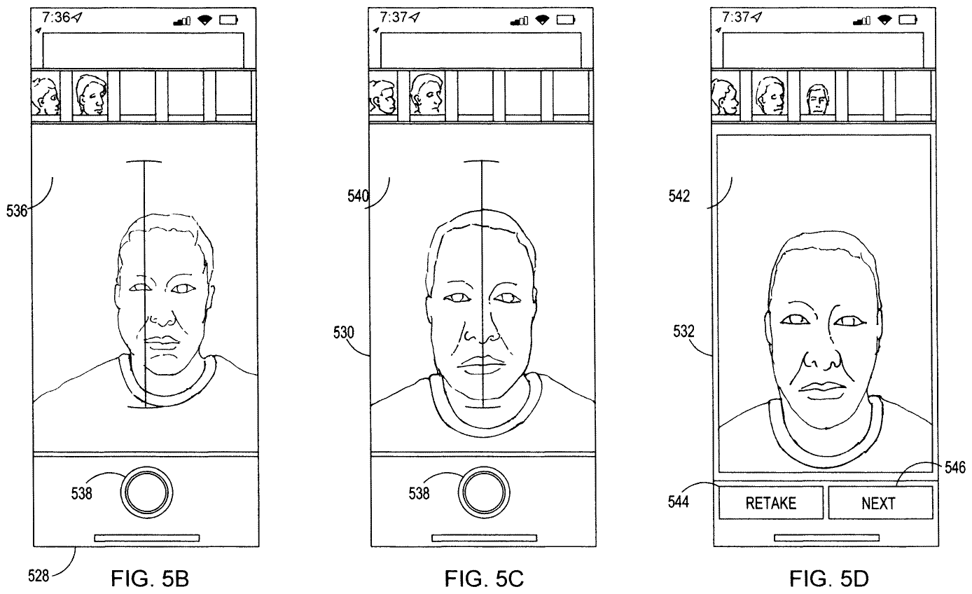

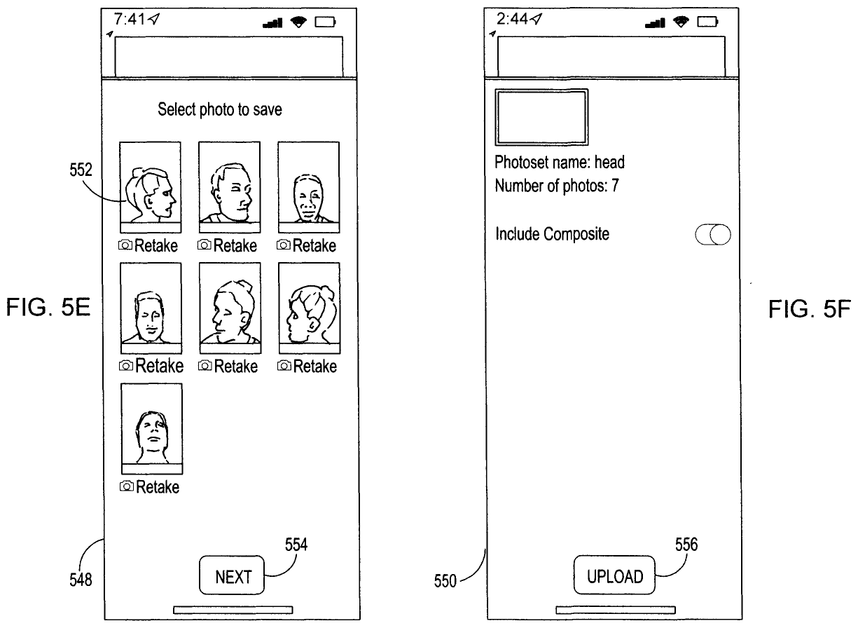

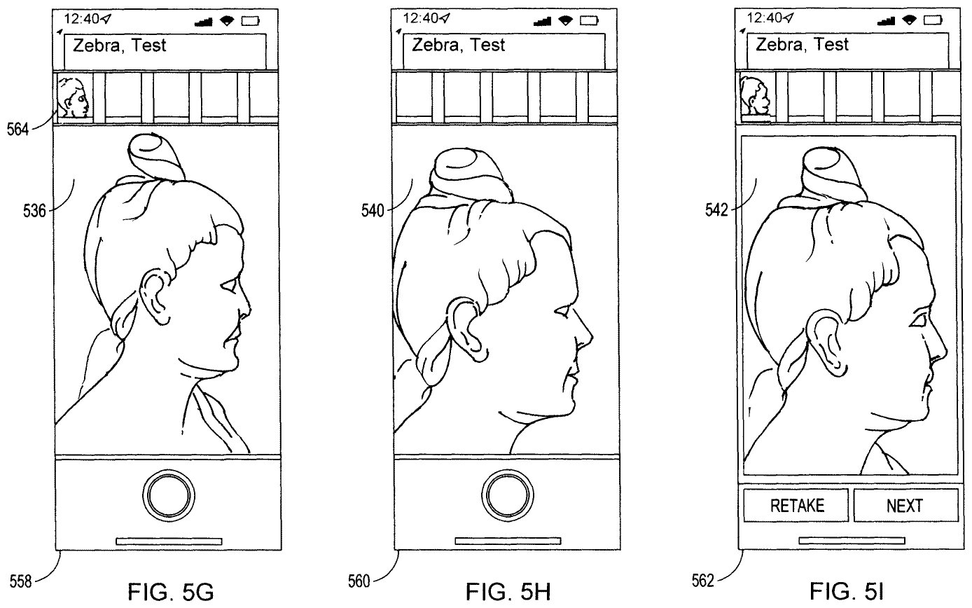

Method 300A may include receiving 304, from the medical provider associated with user device 210, a request for medical images of a patient for a specific procedure. Based on the specific procedure, MIC computing device 204 may determine 306 the required medical images needed from the patient. This may be performed based on one or more standards associated with the specific procedure. The request, along with the images needed, may be forwarded 308 to the patient. In this example, the request may be sent to an application on the patient's mobile device, such as user device 202. The patient may receive a notification (e.g., popup notification, text alert, in-app notification) that further information is needed. The patient may then be prompted by the application to capture 310 one or more images of themselves. In one exemplary embodiment, in view of FIG. 5A, a screen 502 may be displayed showing the multiple views (504, 506, 508, 510, 512, 514, 516, 518, 520, 522, and 524) for the image capture process. The image capture process may be initiated in response to the user selecting button 526 (“Start Capture”). In some embodiments, the application may guide the user through a series of steps to collect the medical image data, as illustrated by example screens 528, 530, 532, 558, 560, and 562 of FIGS. 5B, 5C, 5D, 5G, 5H, and 5I, respectively. For example, one or more graphical indicators, or dynamic digital templates (e.g., arrows, image overlays, dashed lines) may be shown on the interface indicating how the patient should position their face (e.g., looking up/down or left/right). The dynamic digital templates may, in some embodiments, help align the part of the patient (e.g., the patient's nose) that is to be photographed. The dynamic digital templates assist with aligning the patient consistently throughout the image capture process. During image capture, the application may determine whether the user, such as user 534, is within the frame and/or in the appropriate head position. Determining whether a user, or a specific body part (e.g., nose, face), is within the frame or within the bounds of the one or more graphical indicators may be performed using facial detection methods. When the user is in the frame and/or in the appropriate head position, a graphical indicator may be shown to the user. For example, when not in the correct position, a red border or indicator line 536 may be displayed around or in the image preview screen. In one example, the user may be prompted to position themselves so that a right profile view may be captured. In this example, the user may need to align three key elements, forehead, nose and chin (See FIGS. 5G and 5H). Once the user is in the correct position, the border or indicator line may change from red to green (e.g., indicator line 540), and a prompt to “take picture” may be shown. Alternatively, a button on the screen to take a photo, such as button 538 may be enabled, allowing the user to take the picture. In another example embodiment, once the user is within the frame and/or in the correct head position, the image may be captured automatically by the application. In some embodiments, a series of photos may be required, each photo being a different view of the user (e.g., front, side). In one example, the user may be prompted to position themselves so that a right profile view may be captured. In this example, the user may need to align three key elements, forehead, nose and chin (See FIGS. 5G and 5H). Before alignment, the indicator line may be a first color (e.g., red). Once in alignment, the indicator line may change to a second color (e.g., green). After the image is captured, the background may be removed, as shown in screen 562 of FIG. 5I.

During the image capture process, the user may be prompted for each view in succession. Once all views are captured, the image capture process ends. In some embodiments, during the image capture process, the user may select a button 544 to retake an image or, if satisfied with the image captured, select a button 546 to move on to the next image. In some examples, the user may select a photo from a row of thumbnails 564 to retake a photo. After the image is captured, the background may be removed to reduce distraction.

Once all views of the patient are captured, the images may be sent 312 for further image processing. In one embodiment, in view of exemplary screens 548 and 550 of FIGS. 5E and 5F, respectively, the user may be prompted to save one or more photos 552. Once selected, the user may select a “Next” button 554. The next screen, 550, may show a confirmation screen to the user. If satisfied, the user may select the “upload” button 556 to transmit their captured images for image processing. Image processing may be performed on user device 202, MIC computing device 204, or a combination thereof. Image processing may include, for example, background removal (e.g., FIG. 5D, background 542), color correction, image compression, encryption, or the like. Prior to transmission over a network, such as network 212, the captured images may be encrypted to ensure secure file transfer of the images. In some embodiments, the image processing may call for retakes of one or more images. For example, during image processing, it may be determined that one or more images are not in compliance with one or more standards (e.g., image is blurry, does not capture specific body part, image shows the wrong view). Additionally, or alternatively, a medical provider, such as a clinician or plastic surgeon, may review the images and determine that one or more of the images need to be retaken by the patient. In 316, when retakes are needed, a message may be transmitted to the user indicating the need for image retakes. The user may receive the message on their mobile device, such as user device 202, via their application (e.g., via an in-app notification or text message). The application may then repeat the image capture process described above to retake 318 the images needed. Once all photos have been retaken, the image capture process ends and the retaken images are sent for additional image processing 314 (e.g., background removal, color correction, verification). This image retake process may be repeated until all required views are captured and verified. Once all captured images are approved and no more retakes are needed, the captured images are sent 320 to one or more locations. For example, MCI computing device 204 may store the images on database 206, send the images to the medical provider associated with user device 210, stored within the patient's medical record on medical records server 208, or combinations thereof. Additionally, the images may be transmitted back to the patient and stored on the local storage of the user's device.

### Exemplary Method for Clinical and Non-Clinical Image Capture

FIG. 3B depicts an exemplary method 300B for capturing clinical and non-clinical image data of a user. While the example provided is performed within a clinical setting, it is understood that the principles of method 300B may be performed in non-clinical settings (e.g., employee ID badge creation, passport photos, or the like). Method 300B may be performed by a patient's computing device, such as user device 202 (shown in FIG. 2). Further, steps of method 300B may be performed in conjunction with MCI computing device 204, user device 210 and medical provider devices 208, as shown in the exemplary configuration illustrated in FIG. 2. In this example, method 300B provides an example subroutine of method steps 310 and 312 of FIG. 3A with respect to capturing images of a user, such as a patient, for clinical purposes.

Method 300B may include receiving 350 a request to capture images. The request may be received, for example, on a mobile device associated with the patient. In one example, the mobile device may run a software application that interfaces with an electronic medical record associated with the patient. The software application may, in some embodiments, be enabled to capture images of the patient using a camera, such as a camera of the mobile device or another camera communicatively coupled to the mobile device (e.g., a webcam) either through a wired or wireless connection. The patient may receive a notification (e.g., popup notification, text alert, in-app notification) that causes the launching 352 of the software application. The patient may then be prompted by launch 354 a photo session to capture one or more images of themselves. In one exemplary embodiment, in view of FIG. 5A, a screen 502 may be displayed showing the multiple views (504, 506, 508, 510, 512, 514, 516, 518, 520, 522, and 524) for the image capture process. The photo session may be initiated in response to the user selecting button 526 (“Start Capture”).

Method 300B may include determining 356 if one or more digital templates are available that may be used during the photo session. If there are no digital templates (“NO”), then the patient may capture 358 images per the request and the images may be sent for image processing (Block 312). If digital templates are available (“YES”), then the digital templates, at Block 362, may be loaded by the software application for use during the photo session. In some embodiments, digital templates may be stored within libraries accessed by the software application. The libraries may be stored locally (e.g., on the patient's mobile device) or remotely (e.g., storage 206, medical provider devices 208). Digital templates may be associated with certain medical procedures, such as ones identified by the request received by the patient. Additionally, or alternatively, the patient may choose digital templates themselves. For example, if a patient wants to track the progress of their weight loss journey, they may choose a digital template that may guide them through a photo session with a focus on full body shots. In another example, if a medical provider, such as a dermatologist, wants to track the change in appearance of a patient's mole, the patient may be instructed to perform a photo session to capture multiple images and views of the mole and upload them, via the software application, to their electronic medical record on a certain schedule (e.g., once a week, once a month). In some embodiments, digital templates may be created from standardized guidelines issued by governing bodies (e.g., ASPS). The digital templates may be generated programmatically based on the issued standardized guidelines.

In some embodiments, the application may guide the user through a series of steps to collect the medical image data, as illustrated by example screens 528, 530, and 532 of FIGS. 5B, 5C, and 5D, respectively. At Block 364, one or more graphical indicators (e.g., arrows, image overlays, dashed lines) may be shown on the interface indicating how the patient should position their face (e.g., looking up/down or left/right). During image capture, at Block 366, the application may determine whether the user, such as user 534, is within the frame and/or in the appropriate head position. When the user is in the frame and/or in the appropriate head position, a graphical indicator may be shown to the user. For example, at Block 364, when not in the correct position, a red border or indicator line 536 may be displayed around or in the image preview screen. Once the user is in the correct position, at Block 368, the border or indicator line may change from red to green (e.g., indicator line 540), and a prompt to “take picture” may be shown. Alternatively, a button on the screen to take a photo, such as button 538 may be enabled, allowing the user to take the picture. In another example embodiment, once the user is within the frame and/or in the correct head position, the image may be captured automatically by the application. In some embodiments, a series of photos may be required, each photo being a different view of the user (e.g., front, side). The user may be prompted for each view in succession. Once all views are captured, the image capture process ends. In some embodiments, during the image capture process, the user may select a button 544 to retake an image or, if satisfied with the image captured, select a button 546 to move on to the next image. Once all views of the patient are captured, the images may be sent 312 for further image processing.

### Exemplary Multifunction Device

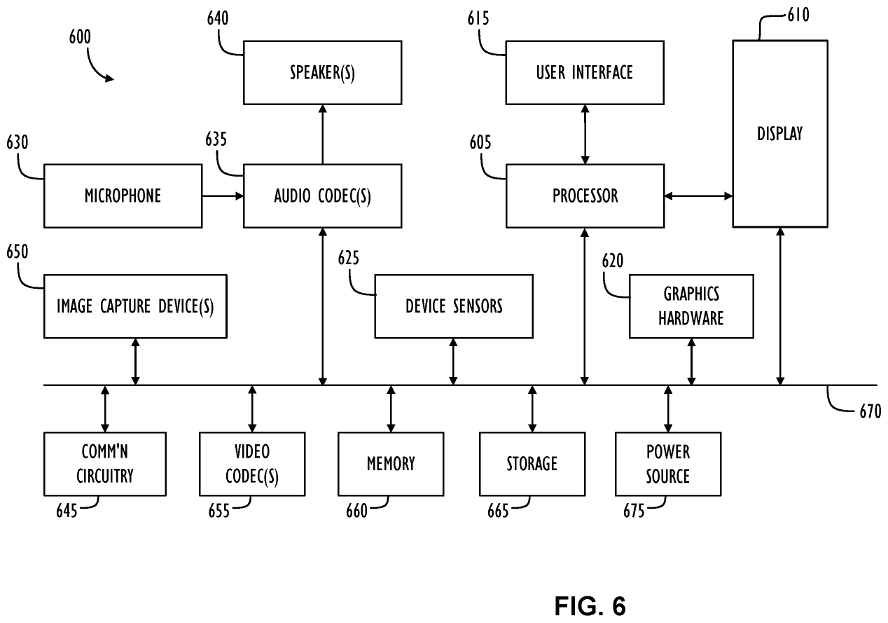

Referring now to FIG. 6, a simplified functional block diagram of illustrative multifunction device 600 is shown according to one embodiment. Multifunctional device 600 may show representative components, for example, for devices of MCI Computing Device 204, user devices 202 and 210, and medical record servers 208 of FIG. 2. Multifunction electronic device 200 may include processor 605, display 610, user interface 615, graphics hardware 620, device sensors 625 (e.g., proximity sensor/ambient light sensor, accelerometer and/or gyroscope), microphone 630, audio codec(s) 635, speaker(s) 640, communications circuitry 645, digital image capture circuitry 650 (e.g., including camera system) video codec(s) 655 (e.g., in support of digital image capture unit), memory 660, storage device 665, and communications bus 670. Multifunction electronic device 600 may be, for example, a digital camera or a personal electronic device such as a personal digital assistant (PDA), personal music player, mobile telephone, a wearable device (e.g., VR headset, smart watch), a tablet computer, or the like.

Processor 605 may execute instructions necessary to carry out or control the operation of many functions performed by device 600 (e.g., such as the generation and/or processing of images as disclosed herein). Processor 605 may, for instance, drive display 610 and receive user input from user interface 615. User interface 615 may allow a user to interact with device 600. For example, user interface 615 can take a variety of forms, such as a button, keypad, dial, a click wheel, keyboard, display screen and/or a touch screen. Processor 605 may also, for example, be a system-on-chip such as those found in mobile devices and include a dedicated graphics processing unit (GPU). Processor 605 may be based on reduced instruction-set computer (RISC) or complex instruction-set computer (CISC) architectures or any other suitable architecture and may include one or more processing cores. Graphics hardware 620 may be special purpose computational hardware for processing graphics and/or assisting processor 605 to process graphics information. In one embodiment, graphics hardware 620 may include a programmable GPU.

Image capture circuitry 650 may capture still and/or video images. Output from image capture circuitry 650 may be processed, at least in part, by video codec(s) 655 and/or processor 605 and/or graphics hardware 620, and/or a dedicated image processing unit or pipeline incorporated within circuitry 665. Images so captured may be stored in memory 660 and/or storage 665.

Sensor and camera circuitry 650 may capture still and video images that may be processed in accordance with this disclosure, at least in part, by video codec(s) 655 and/or processor 605 and/or graphics hardware 620, and/or a dedicated image processing unit incorporated within circuitry 650. Images so captured may be stored in memory 660 and/or storage 665. Memory 660 may include one or more different types of media used by processor 605 and graphics hardware 620 to perform device functions. For example, memory 660 may include memory cache, read-only memory (ROM), and/or random access memory (RAM). Storage 665 may store media (e.g., audio, image and video files), computer program instructions or software, preference information, device profile information, and any other suitable data. Storage 665 may include one more non-transitory computer-readable storage mediums including, for example, magnetic disks (fixed, floppy, and removable) and tape, optical media such as CD-ROMs and digital video disks (DVDs), and semiconductor memory devices such as Electrically Programmable Read-Only Memory (EPROM), and Electrically Erasable Programmable Read-Only Memory (EEPROM). Memory 660 and storage 665 may be used to tangibly retain computer program instructions or code organized into one or more modules and written in any desired computer programming language. When executed by, for example, processor 605 such computer program code may implement one or more of the methods described herein.

### Additional Considerations

As will be appreciated based upon the foregoing specification, the above-described embodiments of the disclosure may be implemented using computer programming or engineering techniques including computer software, firmware, hardware or any combination or subset thereof. Any such resulting program, having computer-readable code, may be embodied or provided within one or more computer-readable media, thereby making a computer program product, i.e., an article of manufacture, according to the discussed embodiments of the disclosure. The computer-readable media may be, for example, but is not limited to, a fixed (hard) drive, diskette, optical disk, magnetic tape, semiconductor memory such as read-only memory (ROM), and/or any transmitting/receiving medium such as the Internet or other communication network or link. The article of manufacture containing the computer code may be made and/or used by executing the code directly from one medium, by copying the code from one medium to another medium, or by transmitting the code over a network.

These computer programs (also known as programs, software, software applications, “apps”, or code) include machine instructions for a programmable processor and can be implemented in a high-level procedural and/or object-oriented programming language, and/or in assembly/machine language. As used herein, the terms “machine-readable medium” “computer-readable medium” refers to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The “machine-readable medium” and “computer-readable medium,” however, do not include transitory signals. The term “machine-readable signal” refers to any signal used to provide machine instructions and/or data to a programmable processor.

As used herein, a processor may include any programmable system including systems using micro-controllers, reduced instruction set circuits (RISC), application specific integrated circuits (ASICs), logic circuits, and any other circuit or processor capable of executing the functions described herein. The above examples are examples only and are thus not intended to limit in any way the definition and/or meaning of the term “processor.”

As used herein, the terms “software” and “firmware” are interchangeable and include any computer program stored in memory for execution by a processor, including RAM memory, ROM memory, EPROM memory, EEPROM memory, and non-volatile RAM (NVRAM) memory. The above memory types are examples only and are thus not limiting as to the types of memory usable for storage of a computer program.

In one embodiment, a computer program is provided, and the program is embodied on a computer readable medium. In an example embodiment, the system is executed on a single computer system, without requiring a connection to a sever computer. In a further embodiment, the system is being run in a Windows® environment (Windows is a registered trademark of Microsoft Corporation, Redmond, Wash.). In yet another embodiment, the system is run on a mainframe environment and a UNIX® server environment (UNIX is a registered trademark of X/Open Company Limited located in Reading, Berkshire, United Kingdom). The application is flexible and designed to run in various different environments without compromising any major functionality. In some embodiments, the system includes multiple components distributed among a plurality of computing devices. One or more components may be in the form of computer-executable instructions embodied in a computer-readable medium. The systems and processes are not limited to the specific embodiments described herein. In addition, components of each system and each process can be practiced independent and separate from other components and processes described herein. Each component and process can also be used in combination with other assembly packages and processes.

As used herein, an element or step recited in the singular and preceded by the word “a” or “an” should be understood as not excluding plural elements or steps, unless such exclusion is explicitly recited. Furthermore, references to “example embodiment” or “one embodiment” of the present disclosure are not intended to be interpreted as excluding the existence of additional embodiments that also incorporate the recited features.

Although specific embodiments of the present invention have been described, it will be understood by those of skill in the art that there are other embodiments that are equivalent to the described embodiments. Accordingly, it is to be understood that the invention is not to be limited by the specific illustrated embodiments, but only by the scope of the appended claims.

What is claimed is:

1. A medical image capture (MIC) computing device capturing standardized face photographs for a plurality of medical providers and a plurality of patients, the MIC computing device comprising one or more processors, the one or more processors configured to:
coordinate access to one or more medical records between one or more medical record servers and a medical provider of the plurality of medical providers, wherein each of the one or more medical records is associated with each of the plurality of patients;
receive, from a computing device associated with the medical provider, a first request for image data of a patient of the plurality of patients;
identify, based on the first request, one or more views required in accordance with a procedure identified by the first request;
transmit, to a computing device of the patient, a second request for one or more photographs in accordance with the one or more views; and
receive, from the computing device of the patient, captured image data of the patient.

2. The MCI computing device of claim 1, wherein the one or more processors are further configured to:
process the captured image data to verify that the captured image data is in compliance with one or more standards.

3. The MCI computing device of claim 2, wherein the one or more processors are further configured to:
in response to the captured image data not being in compliance with the one or more standards, transmit, to the computing device of the patient, a third request to retake one or more photographs.

4. The MCI computing device of claim 2, wherein the one or more processors are further configured to:
in response to the captured image data being in compliance with the one or more standards, upload the captured image data to an electronic medical record of the patient.

5. The MCI computing device of claim 2, wherein the one or more processors are further configured to:
in response to the captured image data being in compliance with the one or more standards, transmit the captured image data to the computing device associated with the medical provider.

6. The MCI computing device of claim 1, wherein the captured image data comprises facial image data of the patient.

7. The MCI computing device of claim 1, wherein the one or more views are associated with a plastic surgery operation identified by the medical provider.

8. A non-transitory computer-readable medium comprising instructions that when executed by one or more processors are configured to implement:
coordinating access to one or more medical records between one or more medical record servers and a medical provider of the plurality of medical providers, wherein each of the one or more medical records is associated with each of the plurality of patients;
receiving, from a computing device associated with the medical provider, a first request for image data of a patient of the plurality of patients;
identifying, based on the first request, one or more views required in accordance with a procedure identified by the first request;
transmitting, to a computing device of the patient, a second request for one or more photographs in accordance with the one or more views; and
receiving, from the computing device of the patient, captured image data of the patient.

9. The non-transitory computer-readable medium of claim 8, wherein the instructions when executed by the one or more processors are further configured to implement:
processing the captured image data to verify that the captured image data is in compliance with one or more standards.

10. The non-transitory computer-readable medium of claim 9, wherein the instructions when executed by the one or more processors are further configured to implement:
in response to the captured image data not being in compliance with the one or more standards, transmitting, to the computing device of the patient, a third request to retake one or more photographs.

11. The non-transitory computer-readable medium of claim 9, wherein the instructions when executed by the one or more processors are further configured to implement:
in response to the captured image data being in compliance with the one or more standards, uploading the captured image data to an electronic medical record of the patient.

12. The non-transitory computer-readable medium of claim 9, wherein the instructions when executed by the one or more processors are further configured to implement:
in response to the captured image data being in compliance with the one or more standards, transmitting the captured image data to the computing device associated with the medical provider.

13. The non-transitory computer-readable medium of claim 8, wherein the captured image data comprises facial image data of the patient.

14. The non-transitory computer-readable medium of claim 8, wherein the one or more views are associated with a medical intervention identified by the medical provider.

15. A method for capturing standardized face photographs for a plurality of medical providers and a plurality of patients, comprising:
coordinating access to one or more medical records between one or more medical record servers and a medical provider of the plurality of medical providers, wherein each of the one or more medical records is associated with each of the plurality of patients;
receiving, from a computing device associated with the medical provider, a first request for image data of a patient of the plurality of patients;
identifying, based on the first request, one or more views required in accordance with a procedure identified by the first request;
transmitting, to a computing device of the patient, a second request for one or more photographs in accordance with the one or more views; and
receiving, from the computing device of the patient, captured image data of the patient.

16. The method of claim 15, further comprising:
processing the captured image data to verify that the captured image data is in compliance with one or more standards.

17. The method of claim 16, further comprising:
in response to the captured image data not being in compliance with the one or more standards, transmitting, to the computing device of the patient, a third request to retake one or more photographs.

18. The method of claim 16, further comprising:
in response to the captured image data being in compliance with the one or more standards, uploading the captured image data to an electronic medical record of the patient.

19. The method of claim 16, wherein the one or more processors are further configured to:
in response to the captured image data being in compliance with the one or more standards, transmitting the captured image data to the computing device associated with the medical provider.

20. The method of claim 15, wherein the captured image data comprises facial image data of the patient.

21. The method of claim 15, wherein the one or more views are associated with a plastic surgery operation identified by the medical provider.
